(19) United States
(12) Plant Patent Application Publication
(10) Pub. No.: US 2004/0006802 P1
(43) Pub. Date: Jan. 8, 2004

(54) CRAPE MYRTLE PLANT NAMED 'WHIT VI'

(76) Inventor: Carl Whitcomb, Stillwater, OK (US)

Correspondence Address: STREETS & STEELE, 13831 NORTHWEST FREEWAY, SUITE 355, HOUSTON, TX, 77040, US

(21) Appl. No.: 10/189,786

(22) Filed: Jul. 3, 2002

(51) Int. Cl. A01H 5/00

(52) U.S. Cl. PLT/252

(57) ABSTRACT

A new and distinct variety of crapemyrtle, Lagerstroemia indica, which is characterized by a dense, upright growth habit; leathery leaves which emerge crimson and change to dark green; low seed production (plant is semi-sterile); inflorescences which are 4 to 14 inches tall and 4 to 12 inches wide; flower buds which are green to crimson; and flowers which are white with transition to light crimson. The new variety is also characterized by a long bloom period.

### BACKGROUND OF THE INVENTION

The present invention relates to a new and distinct variety or cultivar of the ornamental shrub, Lagerstroemia indica, commonly known as crapemyrtle.

The crapemyrtle shrub is native to eastern China and was introduced into North America in the late 1700's. Since then, this popular ornamental shrub has been grown extensively across the southern half of the continent. Over the years, seedlings of Lagerstroemia indica or hybrids between L. indica and L. fauriei have been selected for various growth forms, flower colors, or other features and propagated asexually. An assortment of methods has been utilized in attempting to develop improved varieties of crapemyrtle and several have had U.S. Plant Patents issued.

For example, U.S. Plant Pat. No. 4,182, U.S. Plant Pat. No. 4,183, U.S. Plant Pat. No. 4,184, and U.S. Plant Pat. No. 4,185 disclose and claim a series of four new varieties of Lagerstroemia indica produced by crossing previously known varieties. Each of these varieties was characterized as having a weeping growth habit at maturity. U.S. Plant Pat. No. 5,302 also discloses a new variety of crapemyrtle exhibiting a weeping growth habit at maturity.

U.S. Plant Pat. Nos. 6,365 and 6,383 disclose varieties of crapemyrtle derived from seedlings treated with a mutation-inducing chemical. U.S. Plant Pat. Nos. 10,296 and 10,297 and 10,319 and 11,312 and 11,342 also disclose new varieties of crapemyrtle derived from seedlings where one or more of the parents were treated with a mutation-inducing chemical.

It is generally known that ethyl methane sulfonate (EMS), sodium azide, and coichicine are capable of producing plant mutations. Since one or more of the grandparents of the new variety of crapemyrtle were treated with these compounds, the present invention may be such a mutant.

### SUMMARY OF THE INVENTION

The present invention involves the discovery of a new and distinct variety of crapemyrtle, Lagerstroemia indica, which has been given the cultivar name ‘Whit VI’ and is characterized by a vigorous, broad, upright growing, large shrub or small tree with moderate branching that may reach a height of 10 to 15 feet at maturity. Leaves of the plant emerge crimson and slowly change to dark green with age with mid-veins and backs retaining some crimson color when leaves are in full sun. Mature leaves are larger and more leathery than is typical of the species. Twigs of current season's growth are distinctly crimson unless shaded by other leaves or branches. Inflorescences are 4 to 14 inches tall and 4 to 12 inches wide. Flower buds are green when shaded but light crimson on any parts exposed to full sun, with the margins of the bud scales crimson. Individual petals are pure white, except at the base, which is distinctly, light crimson. Flowers that open during very cool weather in late fall are slightly pinkish throughout. Stamens are prominent and bright yellow. Old flowers fall from the inflorescence with little discoloration. Flowering generally begins in July in North Central Oklahoma and flowering continues until frost. Seed head production is light during July and August and heavier during September and October.

### BRIEF DESCRIPTION OF THE DRAWINGS

FIG. 1 is a photographic view in full color of the growth and flowing of my new crapemyrtle plant in a 3-gallon container.

FIG. 2 is a photographic view in full color of white flowers with yellow stamens and variable green flower buds with crimson lines along the margins of the bud scales of the plant in FIG. 1.

FIG. 3 is a photographic view in full color of an individual flower with white petal blades and light crimson basal portions and yellow stamens, and a green and crimson flower bud showing crimson lines along the margins of the bud scales of the plant in FIG. 1.

FIG. 4 is a photographic view in full color of crimson new foliage, crimson stems and dark green mature foliage of the plant in FIG. 1.

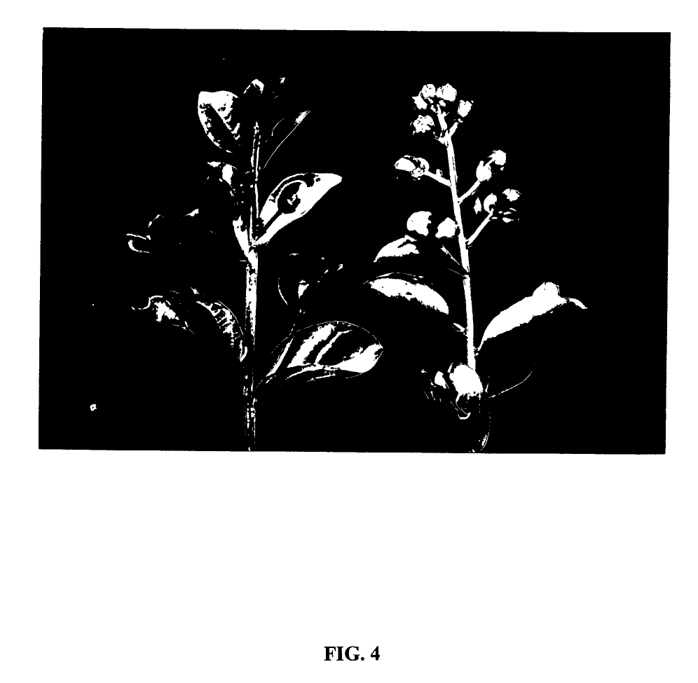

### DETAILED BOTANICAL DESCRIPTION

The new variety of crapemyrtle, which has been, given the cultivar name ‘Whit VI’, was selected from about 2,600 seedlings grown from a 7^{th} generation original seedling plant from when this research began in 1986. The parent seedling selected because of the upright growth, exceptional vigor and prolific lavender flowers that produced many seed pods and viable seeds. The female parent plant grew from seeds that had been treated with a combination of EMS, sodium azide, and colchicine. No attempt was made to control pollination; therefore the pollen could have come from any of the surrounding crapemyrtle seedlings that had also been treated. Seeds were planted in flats. After the seeds germinated, seedlings were transplanted into small containers and mildew was introduced. Seedlings susceptible to mildew or with poor vigor were rouged out. The remainder of the seedlings were planted in rows in an open field for further evaluation.

The new variety was one of 31 seedlings from the population of approx. 2,600 seedlings from this original seedling plant that produced white flowers. The new variety was selected for the crimson new foliage, profusion of flowers in panicles that appear pure white from a distance, dense, upright growth, leathery foliage, and resistance to powdery mildew and resilience following early fall freezes. It may be a mutant seedling arising from the chemical treatment of the female parent seedling.

Softwood cuttings taken from the original parent have been successfully rooted in a medium of peat and perlite under intermittent mist. Subsequent cuttings from plants resulting from previous cuttings have been similarly rooted. Growth, flowering, and flower and foliage color remain consistent with the parent. The plant does produce seeds but viability is low and seedlings are unlike the parent. Asexual reproduction of the new plant by cuttings taken in Stillwater, Okla., has shown that the unique features of this new crapemyrtle are stable and reproduced true to type in successive generations.

A detailed description of the new variety of crapemyrtle follows:

Parentage: Selected from over 150,000 crapemyrtle seedlings grown from a selected seedling parent through seven generations. Seed resulting in the immediate female plant had been treated with a combination of EMS, sodium azide, and colchicine known to induce mutations. The male parent is not specifically known as pollination resulted from natural insect activity among hundreds of seedlings adjacent to the female plant. The new variety was one of 31 seedlings from among the population of approximately 2,600 seedlings that produced white flowers. The thick, leathery leaves and partial sterility are suggestions that this may be a mutant seedling.

Growth: The plant is a vigorous, bushy, upright grower when young. The selected plant reached a height of 4.5 feet one year after a cutting was taken from the parent. Mature height may be 10 to 15 feet. Vegetative growth is rapid in spring and continues until summer when flowering begins. Only modest growth occurs after the onset of flowering. Unlike most crapemyrtle varieties, which produce many vertical stems creating a dense shrub, this new crapemyrtle typically produces 3 to 5 vertical stems with moderate side branches, creating a dense but upright form.

Branches: Current season's stems, exposed to direct light are crimson, greyed-purple group 187-A and CIE 0.410, 0.298, 3.4 (color notations from the Royal Horticulture Society colour chart, 1966 and the Comission International de l'Eclairage, 1931).

Bark: Bark is typical of L. indica species and is exfoliating.

Foliage: New leaves in the spring emerge crimson, greyed-purple group 187-A and CIE 0.410, 0.298, 3.4 and remain so for several weeks. With leaf maturity and the onset of flowering, leaves are Green group, 139-A and CIE 0.292, 0.400, 6.4. to Green group 139-B and CIE 0.311, 0.417, 16.0 with only faint traces of earlier crimson color. When new growth occurs during summer, leaves are crimson, 187-A and CIE 0.410, 0.298, 3.4, only until they reach mature size, which is a much shorter time relative to spring. Foliage color varies with light intensity and growing conditions. Foliage has been very resistant to powdery mildew. Mature leaves are thick and leathery and larger than the species average.

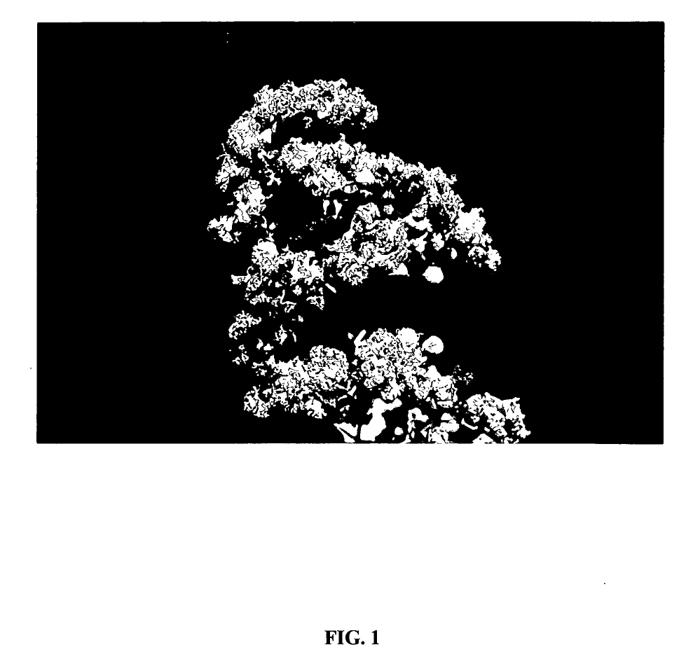

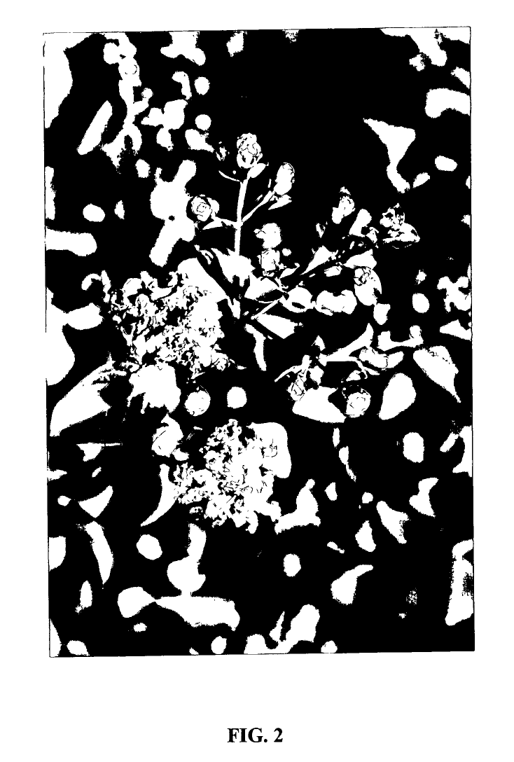

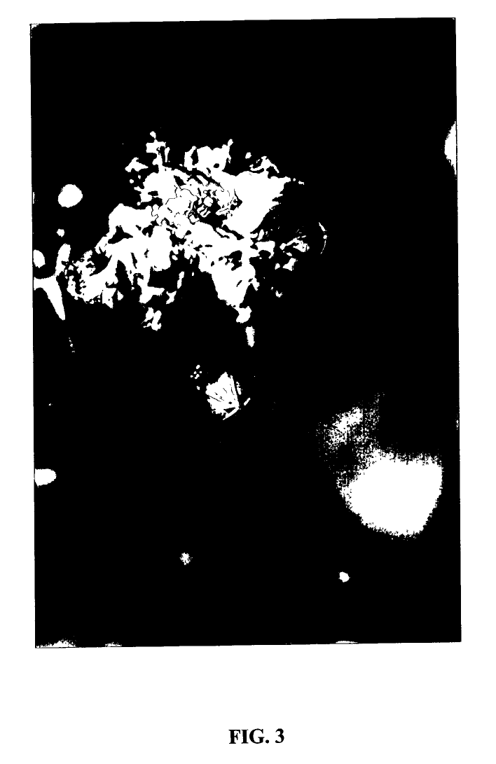

Flowers: Blades of Individual petals (expanded apical portion) are white, white group 155-D and CIE 0.319, 0.330, 88.6, with transition to light crimson, greyed-purple group 185-C and CIE 0.417, 0.293, 16.7, at the claw (the narrowed stalk-like basal portion of the petal) during the summer and early fall. In late fall after temperatures have dropped to near the freezing point and light intensity is low, any late opening flowers are a faint pink, approx. greyed-purple group 186-D and CIE 0.344, 0.298, 44.9. Flower petals open fully, exposing distinct yellow stamens. (FIGS. 1, 2 and 3). As the flowers age they typically fall cleanly from the inflorescence without discoloring or becoming unattractive. Unopened flower buds are variable, slight crimson on sides exposed to direct light and mostly light green on shaded sides, with distinct crimson margins of the bud scales, greyed-purple group 187-B and CIE 0.479, 0.296, 5.1 (FIG. 3). Inflorescences are panicles 4 to 14 inches tall and 4 to 12 inches wide (FIG. 1). The stocks of inflorescences exposed to full sun are crimson, greyed-purple group, 187-A and CIE 0.410, 0.298, 3.4. The new variety begins flowering in July and continues into October in North Central Oklahoma. This flowering period is longer than most seedlings and cultivars of crapemyrtle. Flowering continues during periods of intense and prolonged heat as long as moisture is available. The plant is semi-sterile which appears to make the blooms last longer than is typical of L. indica species. As with other crapemyrtle plants, blooms each have six petals per bloom. Bloom diameter varies with environmental conditions. Because the plant is semi-sterile, few viable seeds are produced. The seed pods that form are typical of L. indica species.

Cold Hardiness: The new variety of crapemyrtle has withstood temperatures of 0° F. with no injury. The top of the parent as well as asexually propagated offspring were killed in October 2000 when, on October 3, the temperature was 92° F., then on October 9, dropped to 23° F. With the arrival of spring 2001, all plants produced vigorous growth and were flowering by early July. This was a distinct contrast to many other seedlings under evaluation that were killed entirely or were very slow to recover from the cold injury.

I claim:

1. A new and distinct variety of crapemyrtle plant, substantially as illustrated and described.
